(19) United States
(12) Patent Application Publication
(10) Pub. No.: US 2020/0032192 A1
(43) Pub. Date: Jan. 30, 2020

(54) CULTURE DEVICE AND CELL CULTURE METHOD USING SAME

(71) Applicant: BOE TECHNOLOGY GROUP CO., LTD., Beijing (CN)

(72) Inventor: Shi ZHOU, Beijing (CN)

(21) Appl. No.: 16/486,414

(22) PCT Filed: Sep. 27, 2018
(86) PCT No.: PCT/CN2018/107937

§ 371 Date: Aug. 15, 2019

(30) Foreign Application Priority Data
Jan. 3, 2018 (CN) 201810004126.4

(51) Int. Cl. C12M 1/34; C12M 1/22; C12M 3/00; C12M 1/00; C12N 5/00

(52) U.S. Cl. CPC C12M 41/12; C12M 23/10; C12N 5/0068; C12M 31/10; C12M 23/46

(57) ABSTRACT

Provided are a culture device and a cell culture method using the culture device. The culture device includes: a temperature sensitive layer and a luminescence emitting structure. The luminescence emitting structure is connected to and overlaps with the temperature sensitive layer, and is configured to emit a cold light that travels through the temperature sensitive layer.

### CROSS REFERENCE TO RELATED APPLICATIONS

The application claims priority to the Chinese patent application No. 201810004126.4, filed on Jan. 3, 2018, the disclosure of which is incorporated herein by reference as part of the application.

### TECHNICAL FIELD

Embodiments of the present disclosure relate to a culture device and a cell culture method using same.

### BACKGROUND

Cell membrane sheet is a research hotspot in the field of tissue engineering in recent years, and is widely applied in the treatment of diseases, such as skin, cornea, heart and periodontitis. The currently used cell membrane sheet preparation device is the temperature-sensitive culture vessel device.

### SUMMARY

An embodiment of the present disclosure provides a culture device, comprising: a temperature sensitive layer; and a luminescence emitting structure connected and overlapped with the temperature sensitive layer and configured to emit a cold light to travel through the temperature sensitive layer.

In an example, the culture device further comprises: a thermal insulating layer disposed between the temperature sensitive layer and the luminescence emitting structure and including a heat insulating material.

In an example, the luminescence structure comprises scattering particles dispersed therein.

In an example, the luminescence structure comprises a luminescent material that emits luminescence after being excited by light or electricity; and the luminescent material comprises at least one selected from the group consisting of fluorescent materials, and phosphorescent materials.

In an example, the temperature sensitive layer is configured to be disengaged from the luminescence structure when the temperature exceeds a preset range.

In an example, the temperature sensitive layer comprises at least one selected from the group consisting of poly N-tetrahydrofurfuryl acrylamide, poly N-n-propyl acrylamide, and poly N-isopropyl acrylamide.

In an example, the culture device further comprises a vessel body. The vessel body comprises a bottom wall and a side wall; an accommodating space is encircled by the bottom wall and the side wall; and the temperature sensitive layer is disposed in the accommodating space.

In an example, the luminescence structure comprises: a light guide plate (LGP) overlapped with the temperature sensitive layer and disposed in the accommodating space; and a light source disposed on an outside of the vessel body.

In an example, the luminescence structure comprises a plurality of luminescence LED light sources in the accommodating space, the plurality of luminescence LED light sources being arranged in a matrix in multiple rows and multiple columns, and overlapped with the temperature sensitive layer.

In an example, the culture device further comprises an electrode wiring layer arranged in the vessel body and disposed on a side of the luminescence structure facing away from the temperature sensitive layer.

In an example, the culture device further comprises a temperature regulating layer disposed on the side wall of the vessel body.

In an example, the culture device further comprises a fixing element disposed on the outside of the vessel body and configured to fix the culture vessel device.

In an example, the fixing element comprises at least two positioning blocks, each positioning block being provided with a positioning slot.

In an example, the fixing element is a positioning rack; and the vessel body is disposed in the positioning rack.

In an example, the temperature regulating layer is disposed on the inner wall of the positioning rack.

In an example, the culture device further comprises an image recording device and a display unit electrically connected with each other. The image recording device is configured to acquire an image of a surface of the temperature sensitive layer facing away from the luminescence structure; and the display unit is configured to display the image acquired by the image recording device.

In an example, the culture device further comprises a temperature sensor and an alarm device electrically connected with each other. The temperature sensor is configured to measure a temperature in the accommodating space of the vessel body; and the alarm device is configured to give an alarm according to the temperature measured by the temperature sensor.

Another embodiment of the present disclosure provides a cell culture method using any of the culture devices, comprising: placing cells to be cultured on a side of the temperature sensitive layer facing away from the luminescence structure; and emitting luminescence to travel through the temperature sensitive layer and irradiating the cells to be cultured by the luminescence structure.

### BRIEF DESCRIPTION OF THE DRAWINGS

A brief description will be given below to the accompanying drawings of the embodiments to provide a more clear understanding of the technical proposals of the embodiments of the present disclosure. Apparently, the drawings described below only involve some embodiments of the present disclosure but are not intended to limit the present disclosure.

FIG. 1 is a schematically structural sectional view of a culture vessel device provided by an example of the embodiment of the present disclosure;

FIG. 2 is a schematically structural sectional view of a culture vessel device provided by another example of the embodiment of the present disclosure;

FIG. 3 is a schematically structural sectional view of a culture vessel device provided by another example of the embodiment of the present disclosure;

FIG. 4 is a schematically external structure diagram of the culture vessel device provided by an embodiment of the present disclosure;

FIG. 5 is a schematically external structure diagram of the culture vessel device provided by an embodiment of the present disclosure; and

FIG. 6 is a schematically external perspective structure diagram of the culture vessel device provided by an embodiment of the present disclosure.

### DETAILED DESCRIPTION

In order to make objects, technical schemes and advantages of the embodiments of the present disclosure clearer, the technical schemes of the embodiments will be described in a clearly and completely way in connection with the drawings related to the embodiments of the disclosure. Apparently, the described embodiments are just a part but not all of the embodiments of the disclosure. Based on the described embodiments herein, one of ordinary skill in the art can obtain other embodiment(s), without any inventive work, which shall be within the scope of the disclosure.

In the process of culturing a cell membrane sheet, a temperature-sensitive material is used as a base layer for the later acquisition and collection of the membrane. As the temperature-sensitive material is extremely sensitive to the temperature change, if ordinary light source is adopted, more heat will be generated, which may adversely affect the temperature-sensitive material. In addition, the sampling of the actual culture solution can often only detect the index change in the culture solution, e.g., pH value, dissolved oxygen concentration, carbon dioxide concentration, lactic acid concentration, etc., but it is difficult to effectively detect important indicators of cell membrane sheets, such as area, thickness, uniformity and flatness of the cell membrane sheet. If a sensor is directly disposed at the bottom of a culture vessel, due to the barrier of the temperature-sensitive material, it may be difficult to effectively measure the parameter change of the cell membrane sheet. So, it is difficult for the cell culture device in the prior art to realize the real-time monitoring of the culture process of the cell membrane sheet.

Embodiments of the present disclosure provide a culture vessel device capable of effectively controlling the temperature in the process of culturing the cell membrane sheet, and a culture method of the cell membrane sheet. The culture vessel device and the culture method of the cell membrane sheet, provided by the embodiments of the present disclosure, can effectively control the temperature in the culture process of the cell membrane sheet, which is conducive to the effective growth of the cell membrane sheet, and can reduce the adverse effect of the temperature on the growth of the cell membrane sheet. Moreover, the culture vessel device and the culture method of the cell membrane sheet, provided by the embodiments of the present disclosure, can also realize the real-time detection of the growth parameters of the cell membrane sheet and effectively monitor the growth process of the cell membrane sheet.

As shown in FIG. 1, the embodiment provides a culture vessel device, which comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the accommodating space M of the vessel body 101. The luminescence structure 108 is configured to emit a cold light. A spacer layer 107 is disposed between the temperature sensitive layer 106 and the luminescence structure 108.

For instance, a surface of the temperature sensitive layer 106 facing away from the luminescence structure 108 is configured to direct contact cells to be cultured.

Herein, the ‘cold light’ refers to visible light emitted from non-incandescent sources at low temperature, e.g., fluorescent light, phosphorescent light, bioluminescent light or triboluminescent light. Because there is no obvious amount of infrared light, the heating effect is very small.

The spacer layer 107, for instance, is a thermal insulating layer including heat an insulating material with the thermal conductivity of less than 0.2 W/(m·K). The temperature sensitive layer 106, the spacer layer 107 and the luminescence structure 108 are overlapped with each other and combined together. In another example, the spacer layer 107 can be omitted, and the temperature sensitive layer 106 and the luminescence structure 108 are overlapped with each other and combined together.

As shown in FIG. 1, a cell membrane sheet to be cultured 105 is placed on the temperature sensitive layer 106, and the temperature sensitive layer 106 is configured as a base layer for the growth of the cell membrane sheet, so as to provide convenience for the later acquisition and collection of the cell membrane sheets. Compared with common thermal light sources, the luminescence structure 108 in the embodiment emits a cold light towards the cell membrane sheets, as less heat is emitted, the adverse effect of the light source on the growth of the cell membrane sheet can be reduced. The arrangement of the spacer layer 107 effectively reduces the effect of the heat generated during the emission of the luminescence structure on the internal ambient temperature of the culture vessel device. In this way, the culture vessel device provided by the embodiment can effectively control the temperature in the culture process of the cell membrane sheet and be conducive to the effective growth of the cell membrane sheet.

The embodiment of the present disclosure provides a culture vessel device, which, for instance, as shown in FIG. 1, comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the accommodating space M of the vessel body 101; the luminescence structure 108 is configured to emit a cold light; and a spacer layer 107 is disposed between the temperature sensitive layer 106 and the luminescence structure 108. The luminescence structure 108 includes scattering particles S, and the scattering particles are uniformly dispersed in the luminescence structure 108 and configured to uniformly scatter a cold light source emitted from the luminescence structure 108.

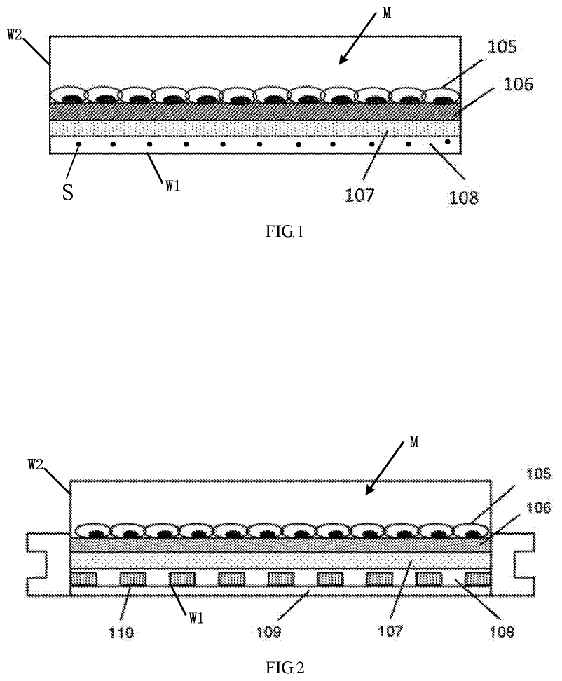

The embodiment of the present disclosure provides a culture vessel device, which, as shown in FIG. 2, comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the accommodating space M of the vessel body 101; the luminescence structure 108 is configured to emit a cold light; and a spacer layer 107 is disposed between the temperature sensitive layer 106 and the luminescence structure 108. As shown in FIG. 2, the luminescence structure 108 includes a plurality of cold light LED sources 110, and the cold light LED sources 110 are arranged in a matrix in the luminescence structure 108. The cold light LED sources with less heat generated compared with the common light source are utilized to uniformly emit cold light sources towards the cell membrane sheet, so as to reduce the adverse effect of the light sources on the growth of the cell membrane sheet.

Optionally, scattering particles S are dispersed in the luminescence structure 108 in intervals. For instance, the scattering particles are all disposed right over the LED light sources 110, and no scattering particles are disposed right over gaps of the LED light sources 110. In this way, the cold light emitted from different areas of the luminescence structure 108 can also be uniformly irradiated towards the cell membrane sheet to be cultured 105.

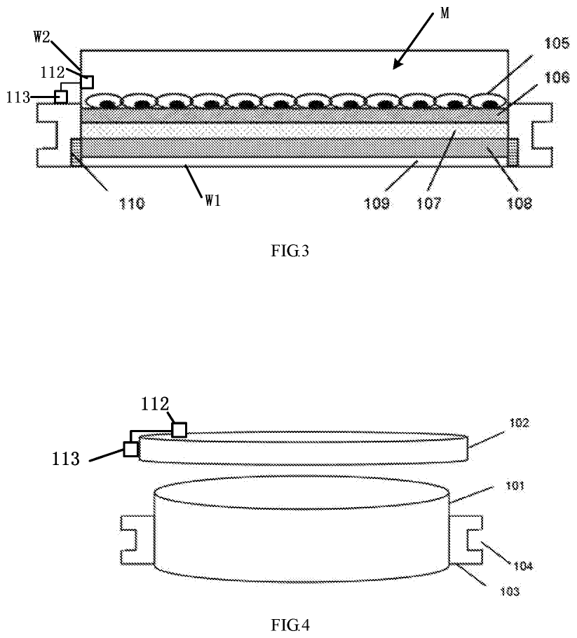

The embodiment of the present disclosure provides a culture vessel device, which, as shown in FIG. 3, comprises a vessel body 101. The vessel body includes a bottom wall W1 and a side wall W2. An accommodating space M is encircled by the bottom wall W1 and the side wall W2. One part of the temperature sensitive layer 106 and one part of the luminescence structure 108 are disposed in the accommodating space M of the vessel body 101. The luminescence structure 108 is configured to emit a cold light. A spacer layer 107 is disposed between the parts of the temperature sensitive layer 106 and the luminescence structure 108. As shown in FIG. 3, the luminescence structure 108, for instance, includes cold light LED sources 110 and an LGP 108′. At least two cold light LED sources 110 are disposed on the outside of the vessel body 101. The LGP 108′ is disposed in the internal accommodating space M of the vessel body 101. The cold light LED source 110 and the LGP 108′ in the vessel body are at the same level. For instance, a surface of the LGP 108′ closest to the temperature sensitive layer 106 is parallel and level to a surface of the cold light LED source 110 closest to the temperature sensitive layer 106. When the cold light LED source 110 emits a cold light, the cold light is irradiated into the LGP 108′, and the cold light emitted by the cold light source is emitted towards a growth layer of the cell membrane sheet through the LGP 108′. The arrangement of the cold light LED source 110 on the outside of the vessel body 101 can further reduce the effect of the heat generated in the vessel body by the cold light LED source 110 on the growth environment of the cell membrane sheet.

Optionally, the dispersion density of the scattering particles on the circumference of the luminescence structure 108 is greater than the dispersion density on the center, which is conducive to well scatter the cold light LED sources on the circumference of the luminescence structure towards the cell membrane sheet to be cultured 105.

The embodiment of the present disclosure provides a culture vessel device, which comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the accommodating space M of the vessel body 101. The luminescence structure 108 is configured to emit a cold light. A spacer layer 107 is disposed between the temperature sensitive layer 106 and the luminescence structure 108. The luminescence structure 108 includes luminescent a material capable of emitting a cold light after being excited by light or by electricity. The cold light luminescent material can emit light within the cold light wavelength range, such as fluorescent light, or phosphorescent light, after being electrically excited. For instance, the cold light luminescent material is one or two of InGaN blue fluorescent material with the emission wavelength of 460 nm and Ca3Sc2Si3O12:Ce green fluorescent material with the emission wavelength of 500 nm. For instance, the cold light emitted by the luminescence structure 108 is a visible light. For instance, the wavelength range of the cold light is 380 nm-560 nm.

The heat given out by the light in the cold light wavelength range is low, so the impact of the heat generated by the light source on the growth of the cell membrane sheet can be effectively reduced. The luminescent materials that emit the cold light may select cold light materials, such as fluorescent luminescent materials, or phosphorescent luminescent materials.

The embodiment of the present disclosure provides a culture vessel device, which comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the accommodating space M of the vessel body 101. The luminescence structure 108 is configured to emit a cold light. A spacer layer 107 is disposed between the temperature sensitive layer 106 and the luminescence structure 108. The temperature sensitive layer 106, for instance, is polymer temperature-sensitive hydrogel or material which can form polymer temperature-sensitive hydrogel when it meets water, and the polymer temperature-sensitive hydrogel can be subjected to volume phase transition with the temperature change. For instance, the polymer temperature-sensitive hydrogel is a thermal expansion temperature-sensitive gel, which is in a shrinkage state when the temperature is lower than the phase transition temperature and is in an expansion state when the temperature is higher than the phase transition temperature. The polymer temperature-sensitive hydrogel may also be thermal shrinkage temperature-sensitive gel, which is in an expansion state when the temperature is lower than the phase transition temperature and is in a shrinkage state when the temperature is higher than the phase transition temperature.

The temperature sensitive layer 106 selects one or more of temperature-sensitive materials including poly N-tetrahydrofurfuryl acrylamide, poly N-n-propyl acrylamide, and poly N-isopropyl acrylamide, and these temperature-sensitive materials are sensitive to the temperature change in the culture vessel. When the culture temperature in the culture vessel device is changed abnormally, namely when the temperature in the culture vessel is deviated from the preset range of the growth temperature of the cell membrane sheet, the temperature sensitive layer 106 is disengaged from the spacer layer 107, so as to avoid the adverse effect of the temperature change on the growth of the cell membrane sheet. For instance, when the temperature is deviated from the primary preset range of the growth temperature of the cell membrane sheet about ±2 Celsius degrees (° C.) to ±5 Celsius degrees (° C.), the temperature sensitive layer 106 is disengaged from the spacer layer 107. For instance, one mode is that: the primary preset range of the culture temperature in the culture vessel is 36 to 38 Celsius degrees (° C.), when the temperature in the culture vessel is deviated from the preset range of the growth temperature of the cell membrane sheet about ±3° C., namely when the temperature in the culture vessel device reaches 40° C., the temperature sensitive layer 106 is disengaged from the spacer layer 107, so as to avoid the adverse effect of the temperature change on the growth of the cell membrane sheet.

The culture vessel device provided by the embodiment further comprises a temperature sensor 112 and an alarm unit 113. For instance, as shown in FIG. 3, the temperature sensor 112 is disposed in the accommodating space M of the vessel body and configured to monitor the temperature condition in the accommodating space M of the vessel body; and the alarm unit 113 is disposed outside of the vessel body 101. The temperature sensor 112 is electrically connected with the alarm unit 113. When the temperature sensitive layer 106 is disengaged from the spacer layer 107 due to the temperature change in the culture vessel, and the temperature sensor 112 transmits an alarm signal about abnormal temperature change to the alarm unit 113, the alarm unit 113 gives an alarm. The alarm unit 113 may be a hummingbird alarm unit or the like, and no specific limitation will be given here. No specific limitation will be given here to the specific mounting position and amount of the alarm unit(s) and the temperature sensor(s).

The embodiment of the present disclosure provides a culture vessel device, which comprises a vessel body 101. The vessel body includes a bottom wall W1 and a side wall W2. An accommodating space M is encircled by the bottom wall W1 and the side wall W2. A temperature sensitive layer 106 and a luminescence structure 108 are disposed in the vessel body 101; the luminescence structure 108 is configured to emit a cold light; and a spacer layer 107 is disposed between the temperature sensitive layer 106 and the luminescence structure 108. As shown in FIG. 2, an electrode wiring layer 109 is also disposed in the accommodating space M of the vessel body of the culture vessel device, and the electrode wiring layer 109 is disposed on the other side of the luminescence structure 108 facing away from the temperature sensitive layer 106. The electrode wiring layer 109 is arranged at the bottom of the vessel body and configured to arrange power lines required by the cold light source layer in the culture vessel.

The embodiment provides a culture vessel device, which comprises a vessel body 101. A temperature sensitive layer 106 and a luminescence structure 108 are disposed in the vessel body 101. The luminescence structure 108 is configured to emit a cold light. A spacer layer 107 is disposed between the temperature sensitive layer 106 and the luminescence structure 108. As shown in FIGS. 2 and 4, fixing elements are disposed on the outside of the culture vessel; the fixing elements are positioning blocks 103; and a positioning slot 104 is disposed in each positioning block 103. The positioning slots 104 are configured to be snapped with other units so as to fix the culture vessel device. At least two positioning blocks are disposed on the outside of the culture vessel and configured to fix the culture vessel device and prevent external force from affecting the environment in the culture vessel and affecting the growth of the cell membrane sheets.

The embodiment of the present disclosure provides a culture vessel device, which comprises a vessel body 101. A temperature sensitive layer 106 and a luminescence structure 108 are disposed in the vessel body 101. The luminescence structure 108 is configured to emit a cold light. A spacer layer 107 is disposed between the temperature sensitive layer 106 and the luminescence structure 108. The culture vessel device is also provided with a temperature regulating layer. The temperature regulating layer selects semiconductor temperature control elements. As shown in FIG. 5, temperature regulating elements 111 are tightly arranged on the outer wall of the culture vessel device and configured to adjust and control the temperature in the culture vessel device, so as to prevent the heat generated by the luminescence structure due to the luminescence structure emitting light for too long time from causing temperature changes in the culture vessel device.

Optionally, the temperature regulating layer may also be tightly arranged on the inner wall of the culture vessel device.

For instance, the temperature regulating layer is, for instance, a semiconductor chilling plate, which achieves an objective of controlling the constant temperature by changing the current magnitude and the current direction.

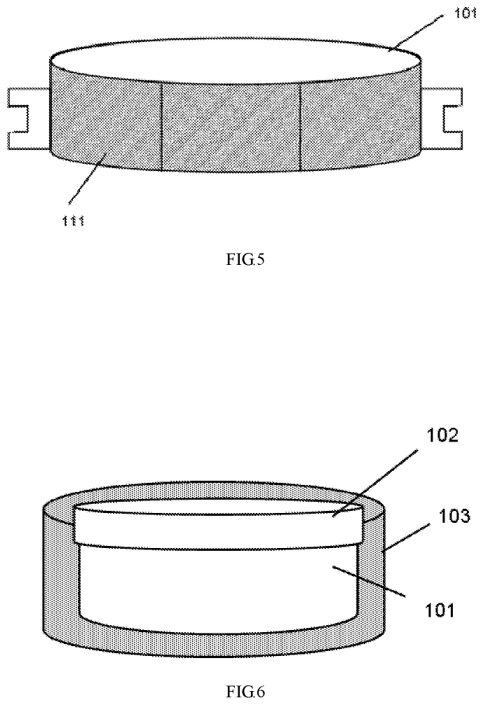

The embodiment provides a culture vessel device, which comprises a vessel body 101. A temperature sensitive layer 106 and a luminescence structure 108 are disposed in the vessel body 101. The luminescence structure 108 is configured to emit a cold light. A spacer layer 107 is disposed between the temperature sensitive layer 106 and the luminescence structure 108. As shown in FIG. 6, a fixing element is disposed on the outside of the vessel body 101, and the fixing element is a culture vessel rack 103. The culture vessel device is also provided with a temperature regulating layer which selects semiconductor temperature control elements. Temperature regulating elements 111 are disposed on the inner wall of the culture vessel rack 103 and tightly arranged along the inner wall of the culture vessel rack 103, so as to avoid the dispersion of the bottom light.

The shape of the culture vessel cooperates with the shape of the culture vessel rack 103 in design, and the shape may be any one selected from round, rectangle, square, diamond, and trapezoid.

The embodiment of the present disclosure provides a culture vessel device, which comprises a vessel body 101 and a vessel cover 102. A temperature sensitive layer 106 and a luminescence structure 108 are disposed in the vessel body 101. The luminescence structure 108 is configured to emit a cold light. A spacer layer 107 is disposed between the temperature sensitive layer 106 and the luminescence structure 108. The temperature sensitive layer 106 and the spacer layer 107, for instance, are both light transmission layers. For instance, as shown in FIG. 4, the culture vessel device further comprises an image recording device 112 and a display unit 113. The image recording device 112 is electrically connected with the display unit 115. The image recording device 114 is configured to acquire an image of a surface of the temperature sensitive layer 106 facing away from the luminescence structure 108. The display unit 115 is configured to display the image acquired by the image recording device 112. For instance, the image recording device is configured to acquire growth processes and growth parameters of the cell membrane sheet 105 to be cultured, and the display unit is configured to display the growth processes and the growth parameters of the cell membrane sheet to be cultured, acquired by the image recording device.

Optionally, the image recording device 112 and the display unit 113 may be mounted on the vessel cover 102, for instance, as shown in FIG. 4; or mounted on the outer wall of the vessel body or the like, and no specific limitation will be given here.

Optionally, the image recording device is a device that can be used for recording images, for example, a CCD image sensor, an infrared image sensor or an electronic microscope.

Optionally, the display unit is a liquid crystal display (LCD), an organic light-emitting display (OLED) or other display devices capable of displaying images.

The monitoring of the growth process of the cell membrane sheet in the prior art is usually to extract a culture solution in the culture vessel, detect the index change in the culture solution, e.g., the parameter values, such as the pH value, the dissolved oxygen concentration, the carbon dioxide concentration and the lactic acid concentration, and then analyze the growth condition of the cell membrane sheet. The process of the cell membrane sheet generation includes adherence of the cell membrane sheet in the latent phase, growth of the cell membrane sheet in the growth phase, and stopping of cell proliferation in the lag phase after the amount of the cells reaches the saturation ratio. However, it is difficult to realize the direct monitoring of the above cell growth process in the prior art.

The growth index of the cell membrane sheet mainly refers to the growth of parameters of the cell membrane sheet, such as the area, the thickness, the uniformity and the flatness of the cell membrane sheet.

After the cell membrane sheet to be cultured 105 is placed on the temperature sensitive layer 106 and adhered to the temperature sensitive layer 106, the temperature sensitive layer 106 acts as a base layer for the generation of the cell membrane sheet, so as to provide convenience for the later acquisition and collection of the cell membrane sheet. The luminescence structure 108 with less heat generated compared with the common light source is utilized to emit a cold light, and the cold light is at least partially emitted towards the direction of the cell membrane sheet through the temperature sensitive layer 106 and the spacer layer 107. On one hand, the cold light can reduce the adverse effect of the light source on the growth of the cell membrane sheet, and the arranged spacer layer 107 can further reduce the impact of the heat generated by the luminescence structure during emission on the temperature of the cell membrane sheet on the temperature sensitive layer 106. In addition, the morphology of the cell membrane sheet can be observed from the other side of the cell membrane sheet irradiated by the cold light facing away from the luminescence structure 108; the image recording device is adopted to record the acquired morphology of the cell membrane sheet through cold light irradiation; an analysis element in the image recording device is adopted to analyze, calculate and obtain the parameters, such as area, thickness, uniformity, and flatness according to the acquired morphology of the cell membrane sheet; the image recording device transmits the acquired morphology of the cell membrane sheet and the parameters, such as area, thickness, uniformity, and flatness obtained after analysis and calculation to the display unit; and at this point, the display unit can display the growth parameters of the cell membrane sheet, such as morphology, area, thickness, uniformity, and flatness, so as to realize the direct real-time detection of the growth process of the cell membrane sheet.

Moreover, the display unit can display a 3D analog picture illustrating the growth process of the cell membrane sheet in the culture vessel device, and other important parameter indexes, through program setting.

Optionally, the display unit is disposed on the outer wall of the vessel body, and the display unit is a transparent display unit.

It is to be understood that description is given in the above embodiment by using the case that the temperature sensitive layer, the luminescence structure and the spacer layer are parallel to the bottom wall W1 of the culture vessel as an example, but it is also possible that the temperature sensitive layer, the luminescence structure and the spacer layer may form a given angle, for instance, the angle is 0-90 degrees. For instance, the mode is that: the temperature sensitive layer, the luminescence structure, and the spacer layer form a 3D structure and are disposed substantially at the center of the culture vessel or at a position close to the inner wall of the culture vessel and perpendicular to the bottom of the culture vessel, in which the temperature sensitive layer of the 3D structure is disposed on the outside of the 3D structure and close to the culture solution, and the luminescence structure is disposed on the inside of the 3D structure, that is to say, as long as the cell membrane sheet to be cultured can be disposed on one side facing away from the temperature sensitive layer and the luminescence structure can be disposed on the other side facing away from the temperature sensitive layer. The following method is also similar.

An embodiment of the present disclosure provides a culture method of a cell membrane sheet, which comprises following operations.

Step1: arranging a temperature sensitive layer and a luminescence structure in a culture vessel, and arranging a spacer layer between the temperature sensitive layer and the luminescence structure.

Step2: adjusting the temperature range of the culture vessel device, and placing the cell membrane sheet to be cultured on a side of the temperature sensitive layer facing away from the luminescence structure.

Step3: using the luminescence structure to emit a cold light towards the direction of the cell membrane sheet, the cold light is at least partially emitted towards the direction of the cell membrane sheet through the temperature sensitive layer and the spacer layer disposed in the culture vessel device.

Moreover, when the temperature in the culture vessel device is deviated from the preset range of the growth temperature of the cell membrane sheet, the temperature sensitive layer is disengaged from the spacer layer.

The temperature range of the culture vessel device is adjusted. After the cell membrane sheet to be cultured 105 is adhered to the temperature sensitive layer 106, the temperature sensitive layer 106 is used as a base layer for the generation of the cell membrane sheet, so as to provide convenience for the later acquisition and collection of the cell membrane sheet.

Compared with the common thermal light source, the luminescence structure 108 in the embodiment with less heat generated emits a cold light towards the cell membrane sheet, because the heat generated by the luminescence structure is less, the adverse effect of the light source on the growth of the cell membrane sheet can be reduced. The arrangement of the spacer layer 107 can effectively reduce the impact of the heat generated by the luminescence structure during emission on the ambient temperature in the culture vessel device. The morphology of the cell membrane sheet can be observed from the other side of the cell membrane sheet irradiated by the cold light facing away from the luminescence structure 108; the image recording device is adopted to record the acquired morphology of the cell membrane sheet through the cold light irradiation; an analysis element in the image recording device is adopted to analyze, calculate, and obtain the parameters, such as area, thickness, uniformity, and flatness according to the acquired morphology of the cell membrane sheet; the image recording device transmits the acquired morphology of the cell membrane sheet and the parameters, such as area, thickness, uniformity, and flatness obtained after analysis and calculation to the display unit; and at this point, the display unit can display the growth parameters of the cell membrane sheet, such as morphology, area, thickness, uniformity, and flatness, so as to realize the direct real-time detection of the growth process of the cell membrane sheet.

The temperature sensitive layer 106 selects one or more of temperature-sensitive materials including poly N-tetrahydrofurfuryl acrylamide, poly N-n-propyl acrylamide and poly N-isopropyl acrylamide, and these temperature-sensitive materials are sensitive to the temperature change in the culture vessel. When the culture temperature in the culture vessel device is changed abnormally, namely when the temperature in the culture vessel is deviated from the preset range of the growth temperature of the cell membrane sheet, the temperature sensitive layer 106 is disengaged from the spacer layer 107, so as to avoid the adverse effect of the temperature change on the growth of the cell membrane sheet. For instance, when the temperature is deviated from the primary preset range of the growth temperature of the cell membrane sheet about ±2 Celsius degrees (° C.) to ±5 Celsius degrees (° C.), the temperature sensitive layer 106 is disengaged from the spacer layer 107. For instance, one mode is that: the primary preset range of the culture temperature in the culture vessel is 36 to 38 Celsius degrees (° C.), when the temperature in the culture vessel is deviated from the preset range of the growth temperature of the cell membrane sheet about ±3° C., namely when the temperature in the culture vessel device reaches 40° C., the temperature sensitive layer 106 is disengaged from the spacer layer 107, so as to avoid the adverse effect of the temperature change on the growth of the cell membrane sheet.

The following points should be noted.

(1) The accompanying drawings involve only the structure(s) in connection with the embodiment(s) of the present disclosure, and other structure(s) can be referred to common design(s).

(2) Without conflicting with each other, elements in one embodiment or in different embodiments can be combined.

The above embodiments are only configured to explain the embodiments of the present disclosure, and are not intended to limit the embodiments of the present disclosure. Various changes and variants can also be made by one of ordinary skill in the art without departing from the spirit and the scope of the embodiments of the present disclosure. Therefore all the equivalent technical proposals are also within the scope of the embodiments of the present disclosure, and the scope of the present disclosure should be defined by the claims.

1. A culture device, comprising:
a temperature sensitive layer; and
a luminescence emitting structure connected and overlapped with the temperature sensitive layer and configured to emit a cold light to travel through the temperature sensitive layer.

2. The culture device according to claim 1, further comprising: a thermal insulating layer disposed between the temperature sensitive layer and the luminescence emitting structure and including a heat insulating material.

3. The culture device according to claim 2, wherein the luminescence structure comprises scattering particles dispersed therein.

4. The culture device according to claim 3, wherein the luminescence structure comprises a luminescent material that emit a cold light after being excited by light or electricity; and the luminescent material comprises at least one selected from the group consisting of fluorescent materials, and phosphorescent materials.

5. The culture device according to claim 4, wherein the temperature sensitive layer is configured to be disengaged from the luminescence structure when the temperature exceeds a preset range.

6. The culture device according to claim 5, wherein the temperature sensitive layer comprises at least one selected from the group consisting of poly N-tetrahydrofurfuryl acrylamide, poly N-n-propyl acrylamide, and poly N-isopropyl acrylamide.

7. The culture device according to claim 6, further comprising a vessel body, wherein the vessel body comprises a bottom wall and a side wall; an accommodating space is encircled by the bottom wall and the side wall; and the temperature sensitive layer is disposed in the accommodating space.

8. The culture device according to claim 7, wherein the luminescence structure comprises:
a light guide plate (LGP) overlapped with the temperature sensitive layer and disposed in the accommodating space; and
a light source disposed on an outside of the vessel body.

9. The culture device according to claim 7, wherein the luminescence structure comprises a plurality of luminescence LED light sources in the accommodating space, the plurality of luminescence LED light sources being arranged in a matrix in multiple rows and multiple columns, and overlapped with the temperature sensitive layer.

10. The culture device according to claim 9, further comprising:
an electrode wiring layer arranged in the vessel body and disposed on a side of the luminescence structure facing away from the temperature sensitive layer.

11. The culture device according to claim 7, further comprising: a temperature regulating layer disposed on the side wall of the vessel body.

12. The culture device according to claim 7, further comprising: a fixing element disposed on the outside of the vessel body and configured to fix the culture vessel device.

13. The culture device according to claim 12, wherein the fixing element comprises at least two positioning blocks, each positioning block being provided with a positioning slot.

14. The culture device according to claim 12, wherein the fixing element is a positioning rack; and the vessel body is disposed in the positioning rack.

15. The culture device according to claim 14, wherein the temperature regulating layer is disposed on the inner wall of the positioning rack.

16. The culture device according to claim 15, further comprising: an image recording device and a display unit electrically connected with each other,
wherein the image recording device is configured to acquire an image of a surface of the temperature sensitive layer facing away from the luminescence structure; and
the display unit is configured to display the image acquired by the image recording device.

17. The culture device according to claim 16, further comprising: a temperature sensor and an alarm device electrically connected with each other, wherein
the temperature sensor is configured to measure a temperature in the accommodating space of the vessel body; and
the alarm device is configured to give an alarm according to the temperature measured by the temperature sensor.

18. A cell culture method using the culture device according to claim 1, comprising:
placing cells to be cultured on a side of the temperature sensitive layer facing away from the luminescence structure; and
emitting luminescence to travel through the temperature sensitive layer and irradiating the cells to be cultured by the luminescence structure.
